(19) United States
(12) Patent Application Publication
(10) Pub. No.: US 2006/0212131 A1
(43) Pub. Date: Sep. 21, 2006

(54) Energy returning prosthetic foot

(76) Inventor: Michael J. Curtis, Green Bay, WI (US)

Correspondence Address: NICHOLAS A. BRANNEN, 104 SOUTH MAIN STREET, SUITE 300, FOND DU LAC, WI, 54935, US

(21) Appl. No.: 11/080,108

(22) Filed: Mar. 15, 2005

(51) Int. Cl. A61F 2/66

(52) U.S. Cl. 623/55; 623/52

(57) ABSTRACT

The present invention relates to a prosthetic foot comprising a rear foot portion having an attachment platform and a heel strike, and a front foot portion having a momentum interrupter and a toe plate. As the foot approaches toe-off, the toe plate flexibly deflects upwards and the momentum interrupter compresses. The amount of returned energy at toe-off is comprised from stored flexural energy of the toe plate and stored expansive energy of the momentum interrupter. The toe plate of the present invention can have a split toe design. Further, mechanically adjustable stiffeners can be incorporated into the design if desired.

### BACKGROUND OF THE INVENTION

1. Field of the Invention

The present invention relates to a prosthetic foot, and more particularly to an energy returning prosthetic foot comprising a momentum interrupter, that is flexible about many axis, and that is efficient at storing and returning energy during use.

2. Description of the Related Art

People frequently are in need of prosthetic limbs as a result of accidents, disease or birth defects. The need for quality prosthetic feet is high. There have been many attempts to make suitable prosthetic feet, each desiring to reach goals of safety, functionality and comfort, among other qualities. The existing prosthetic feet have achieved varying levels of success at attaining each of these stated goals. Further, many strategies have been utilized in designing prosthetic feet. Some designs are relatively noncomplicated, and seek use a simple design. Other designs are complex, and seek to simulate or copy the structure of the human foot.

One prosthetic foot in particular is made by Freedom Innovations and sold under the name Renegade LP. This foot has an uninterrupted component spanning from the ankle to the toe. A second component is extends from the heel to the front of the foot. While this foot may work well for its intended purpose, it is not without some limitations. For example, the amount of energy return at toe-off consists of energy stored during the flexing of the single uninterrupted component spanning from the ankle to the toe. Another limitation of this foot is that there is no way to adjust the amount of flex of the uninterrupted component in order to accommodate for persons having various weights and/or lifestyles.

Another existing prosthetic foot is sold by Ossur under the name Modular III. This foot has one uninterrupted component forming the ankle and spanning to the toe area. A heel component depends rearward from the major component to the heel. This heel component connects to the bottom surface of the major component. A primary advantage of this foot resides in its simplicity. However, there are drawbacks also associated therewith. One limitation is the lack of adjustability. A further limitation is that the amount of energy return at toe-off consists of the energy stored during the flexing of the single uninterrupted component spanning from the ankle to the toe.

A third prosthetic foot is an assembly that is shown in U.S. Pat. No. 6,129,766 to Johnson et al. This patent discloses an ankle member, a heel member pivotally connected to the ankle member, and an elongate metatarsal-toe member having a rear portion underlying a forward portion of the heel member and projecting forwardly from the heel member. This foot has compressible elements incorporated between the pivotally connected members. This foot, being an assembly, is relatively complicated when compared to the previously described prosthetic feet. A further limitation of this foot is that there may be inherent noise problems incorporated into the design of the compressible elements. This is evidenced by the presence, of and the need for, noise abatement features being described in the disclosure.

A fourth prosthetic foot is sold by Otto Bock under the name LuXon Max. This foot shows a relatively flat and uninterrupted component spanning from the ankle location forward to a location above and rearward of the toe location. Distinct heel and toe components are connected to the bottom of the flat component. One limitation with this foot is that the amount of energy return at toe-off consists of the energy stored during the flexing of the single uninterrupted component spanning from the ankle to the toe. Another limitation of this foot is that there is no way to adjust the flexing settings of the single uninterrupted component to accommodate for persons having various weights and/or lifestyles.

Yet another prosthetic foot is shown in U.S. Pat. No. 6,602,295 to Doddroe et al. The foot shown in this patent has a foot plate, a toe spring and a heel spring. The foot plate is an uninterrupted plate that spans from heel to toe.

Another prosthetic foot is shown in U.S. Pat. No. 6,241,776 to Christensen. The foot shown in this patent has a forefoot reinforcement member extending from an attachment section, through a curvilinear spring and arch section, to a toe end. A heel member extends from the arch section to the heel end. The strength and energy return in this foot is due to the flexing of the members. A limitation of this foot is that it is lacking is adjustability.

Another prosthetic foot is shown in U.S. Pat. No. 5,037,444 to Phillips. That patent shows a foot with a forefoot portion and a demountably connected heel portion. At toe-off, the energy return is created from energy stored during the flexing of the forefoot member.

Thus there exists a need for a prosthetic foot that solves these and other problems.

### SUMMARY OF THE INVENTION

The present invention relates to a prosthetic foot that has a rear foot portion and a front foot portion, and advantageously comprises a forefoot momentum interruption component.

According to one embodiment of the present invention, the rear foot portion generally comprises a riser panel, an attachment platform, and a heel strike. The front foot portion generally comprises a momentum interrupter and a toe plate. These components may be integrally formed, and can be made from carbon epoxy composite material. The riser panel can be generally vertically situated in the prosthetic foot. The attachment platform depends rearward from the top of the riser panel, and is provided so that an adapter or other prosthetic component can attach to the prosthetic foot of the present invention. The heel strike is connected to and depends rearward from the bottom of the riser panel, and is generally located below the attachment platform. The momentum interrupter interconnects the toe plate and the riser panel. The toe plate of the present invention can have a split toe design. Further, mechanically adjustable stiffeners can be incorporated into the design whenever desired; such as when increased load situations are anticipated.

According to another embodiment of the present invention, the rear foot portion can comprise the heel strike of a base component, an attachment platform of a momentum interruption component, and a vertical shock absorption component. The front foot portion can comprise a toe portion of the base component, and the momentum interrupter and toe reinforcement components of the momentum interruption component.

In both embodiments, the momentum interrupter provides forefoot momentum interruption. The momentum interrupter compresses when the toe plate flexes upwards to a deflected position during load conditions. At toe off, the amount of returned energy in the foot is derived from the stored flexural energy of the toe plate and the stored expansive energy of the momentum interrupter.

One advantage of the prosthetic foot of the present invention is that it incorporates a momentum interrupter into its design. The momentum interrupter compresses when the person moves to mid-stance, and then further compresses as the person moves towards toe-off. The momentum interrupter then expands at toe-off to linearly help push the prosthetic foot rear portion away from the ground. This energy is in addition to the flexural energy released from the toe plate during toe-off.

A further advantage of the present invention is that there are no moving or rotatably connected parts that may fail over time. Further, since none of the components of the present invention are movingly or rotatably connected to each other, and since none of these components rub against each other during use, the foot is free from undesired noises. There is therefore no need for noise abatement features.

A still further advantage of the present invention is that it is customizable to suit the specific needs of any given person. This is initially accomplished by selecting the appropriate blank foot from a given number of sizes of blank feet. The prosthetic foot can then be reduced from the initial size down to the desired length. Further, a left or right foot can be constructed as desired from a single blank. Still further, toes or toe like projections can be formed as desired to further add to the flexibility of the foot.

A still further advantage yet of the prosthetic foot of the present invention is that the present invention provides support to the person at mid-stance. This is accomplished by flexing of the heel strike due to contact with the ground, flexing of the toe plate due to contact with the ground, flexing of the adapter plate due to the weight of the person and compression of the momentum interrupter. These flexing and compressing components relieve problems that may normally be associated with flat foot, and the energy returned from the heel-strike assists the person in moving from mid-stance to toe-off.

A still further advantage yet of the prosthetic foot of the present invention is that stiffeners can be incorporated into the foot to further increase the adjustability of the prosthetic foot. The stiffeners can be mechanically adjustable stiffeners to provide even further adjustability to the present invention. The stiffeners can be placed between the adapter plate and the heel strike to provide additional support at heel-strike. A stiffener can also be incorporated into the foot to selectively reduce the compressibility of the momentum interrupter. Such stiffeners can be used as desired, such as when the person will be engaging in high impact environments such as playing sports, or when the person is a heavier individual.

Other advantages, benefits, and features of the present invention will become apparent to those skilled in the art upon reading the detailed description of the invention and studying the drawings.

### BRIEF DESCRIPTION OF THE DRAWINGS

FIG. 1 is a side view of a preferred embodiment of the prosthetic foot of the present invention.

FIG. 2 is a partial side view of a preferred embodiment of the attachment platform of the present invention having an adapter attached thereto.

FIG. 3 is a top view of the preferred embodiment shown in FIG. 1.

FIG. 4 is side view of the operation of a preferred embodiment of the prosthetic foot of the present invention at heel-strike.

FIG. 5 is side view of the operation of a preferred embodiment of the prosthetic foot of the present invention at mid-stance.

FIG. 6 is side view of the operation of a preferred embodiment of the prosthetic foot of the present invention at toe-off.

FIG. 7 is a side view of an alternative preferred embodiment of the prosthetic foot of the present invention.

FIG. 8 is a side view of an alternative preferred embodiment of the prosthetic foot of the present invention.

FIG. 9 is a side view of an alternative preferred embodiment of the prosthetic foot of the present invention.

FIG. 10 is a top view of an alternative preferred embodiment of the prosthetic foot of the present invention.

FIG. 11 is a top view of an alternative preferred embodiment of the prosthetic foot of the present invention.

FIG. 12 is a top view of an alternative preferred embodiment of the prosthetic foot of the present invention.

FIG. 13 is a top view of an alternative preferred embodiment of the prosthetic foot of the present invention.

FIG. 14 is a side view of an alternative preferred embodiment of the prosthetic foot of the present invention showing an object under a toe.

FIG. 15 is a cross-sectional side view of a preferred embodiment of the prosthetic foot of the present invention in an intended environment.

FIG. 16 is a perspective view of a further alternative preferred embodiment of a prosthetic foot of the present invention.

FIG. 17 is a side view of the prosthetic foot shown in FIG. 16.

FIG. 18 is a side view of the operation of an alternative preferred embodiment of the prosthetic foot of the present invention at heel-strike.

FIG. 19 is side view of the operation of an alternative preferred embodiment of the prosthetic foot of the present invention at mid-stance.

FIG. 20 is side view of the operation of an alternative embodiment of the prosthetic foot of the present invention at toe-off.

### DETAILED DESCRIPTION OF THE PREFERRED EMBODIMENTS

While the invention will be described in connection with several preferred embodiments, it will be understood that it is not intended to limit the invention to those embodiments. On the contrary, it is intended to cover all alternatives, modifications and equivalents as may be included within the spirit and scope of the invention as defined by the appended claims.

The prosthetic foot 10 of the present invention is preferably made from a carbon epoxy composite material. It will be understood that the present invention is not limited to being constructed of carbon epoxy composite material, and that other resilient materials can be used without departing from the broad aspects of the present invention.

The foot 10 is preferably made in blanks that can be reduced in size to form left and right feet, as desired, and shortened to meet the requirements of a particular person. Several size blanks can be made so that practitioners can select an appropriate blank for a starting point.

The prosthetic foot 10 of the present invention can be made to any desired thickness. Given that the thickness of the material is related to the flexural strength and amount of deflection of any given component of the foot, it is understood that blanks can be constructed having various thicknesses, and that each component within any given foot may be made with a different thickness. Further, it is understood that the thickness of material within any given component of the foot 10 can be tapered to achieve a desired flexural characteristic.

Referring now to FIGS. 2 and 15, it is shown that the prosthetic foot 10 is intended for use with an adapter 6 of a type shown, or to any other type of prosthetic component (not shown) in order to be connected to the remainder of the prosthetic limb. Other types of adapters (not shown) could be provided for connecting directly to the end of a stump. The prosthetic foot 10 can be inserted into a shell 7, and the shell can then be used to contact the ground 5. It will be understood that for sake of clarity, the prosthetic foot 10 of the present invention is hereafter described in some circumstances in direct relation to the ground 5 without the showing of a shell 7.

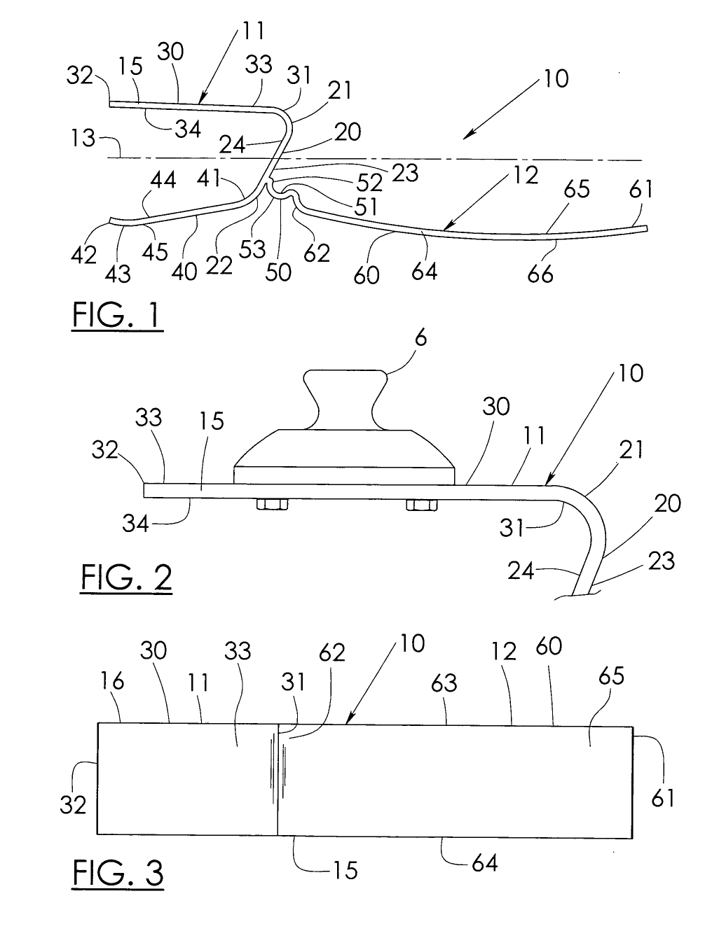

Looking now at FIGS. 1 and 3, one preferred embodiment of the present invention is shown. Accordingly, a foot 10 is provided that is generally comprised of a rear foot portion 11 and a front foot portion 12. The rear foot portion 11, or rear portion, is generally comprised of a riser panel 20, an attachment platform 30 and a heel strike 40. The front foot portion 12 is generally comprised of a momentum interrupter 50 and a toe plate 60. When viewed from above, the prosthetic foot has a right side 15 and a left side 16. The prosthetic foot has a longitudinal axis 13 spanning the length of the foot 10. The longitudinal axis 13 is generally parallel with the ground 5 when the foot 10 rests on the ground during a no load condition.

Looking at FIG. 1, it is shown that a riser panel 20 is provided. The riser panel 20 has a top end 21 and a bottom end 22. The riser panel 20 also has a front surface 23 and a rear surface 24. In the preferred embodiment, the riser panel 20 is a forward leaning riser panel, such that the top end 21 is located forward of the bottom end 22. However, other orientations of the riser panel 20 could be used without departing from the broad aspects of the present invention. The riser panel 20 is flexible along its length.

It is also shown in FIG. 1 that an attachment platform 30 is provided. The attachment platform 30 has a front end 31 and a rear end 32. The attachment platform 30 also has a top surface 33 and a bottom surface 34. The front end 31 of the attachment platform 30 is preferably integrally connected to the top end 21 of the riser panel 20, such that the rear end 32 is distal from the riser panel 20. This connection is preferably a rounded connection wherein sharp edges are eliminated. The attachment platform 30 flexibly depends rearward from the riser panel 20. The attachment platform 30 is preferably angled upward so that the rear end 32 is higher than the front end 31. During a downward load condition, the attachment platform 30 flexes or deflects downward in response to an applied force. In this regard, the rear end 32 deflects down in relation to the front end 31. Conversely, during an upward load condition, the attachment platform 30 deflects upward in response to the applied force. In both types of load conditions, the attachment platform 30 stores and then releases the stored energy when the load condition is ended.

It is shown in FIG. 2 that an adapter 6 can be connected to the attachment platform 20. In the illustrated embodiment, the adapter 6 is bolted to the top surface 33 of the attachment platform 30 with bolts that extend through the attachment platform 30. However, other methods of attachment can be utilized without departed from the broad aspects of the present invention.

Returning again to FIG. 1, it is shown that a heel strike 40 is also provided. The heel strike 40 has a front end 41 and a rear end 42. A lip 43 can be at the rear end 42 of the heel strike 40. The heel strike 40 further has a top surface 44 and a bottom surface 45. The heel strike 40 is preferably integrally connected to the bottom end 22 of the riser panel 20, such that the rear end 42 is distal from the riser panel 20. This connection is preferably a rounded connection wherein sharp edges are eliminated. The heel strike 40 flexibly depends rearward from the riser panel 20. The heel strike 40 preferably flexibly depends downward from the riser panel 20, such that the heel strike rear end 42 is lower than the heel strike front end 41. The lip 43 can be angled with respect to the remainder of the heel strike 40 such that it is close to parallel with the foot longitudinal axis 13 in a no load condition. During a load condition, the heel strike 40 deflects upward in response to an applied force due to the contact with the ground. In this regard, the rear end 42 deflects upward in relation to the front end 41. The heel strike 40 stores and then releases the stored energy when the load condition is ended.

A momentum interrupter 50 is advantageously provided according to the present invention. A preferred momentum interrupter 50 has a front 51, a rear 52 and a bottom 53. In this regard, the momentum interrupter 50 generally has a U-shape. However, other shapes can be provided without departing from the broad aspects of the present invention. The momentum interrupter 50 is preferably integrally connected to the riser panel 20, wherein the momentum interrupter rear 52 is integral with the riser panel front surface 23. In the illustrated embodiment, the momentum interrupter 50 is shown integrally connected near the bottom end 22 of the riser panel 20. However, other orientations can exist without departing from the broad aspects of the present invention. The momentum interrupter has a length between the front 51 and rear 52. During a load condition, the momentum interrupter front 51 compresses towards the momentum interrupter rear 52, and the momentum interrupter length is decreased. This compression preferably occurs along a compression axis. The momentum interrupter 50 stores and then releases the stored energy when the load condition is ended.

Calling attention again to FIGS. 1 and 3, a toe plate 60 is provided. The toe plate 60 has a front end 61 and a rear end 62. The rear end 62 is preferably an upwardly curved rear end. The toe plate 60 further has a left side 63 and a right side 64, and has a top surface 65 and a bottom surface 66. The toe plate 60 is preferably integrally connected to the momentum interrupter 50. In this regard, the upwardly curved rear end 62 is integrally connected to the front 51 of the momentum interrupter 50, and the front end is distal from the momentum interrupter 50. The toe plate preferably is slightly upwardly curved along its length. The toe plate 60 flexibly depends forwardly and preferably slightly downwardly from the momentum interrupter 50. During a load condition, the front end 61 of the toe plate 60 flexibly deflects upward in relation to the rear end 62. The toe plate 60 stores and then releases the stored energy when the load condition is ended.

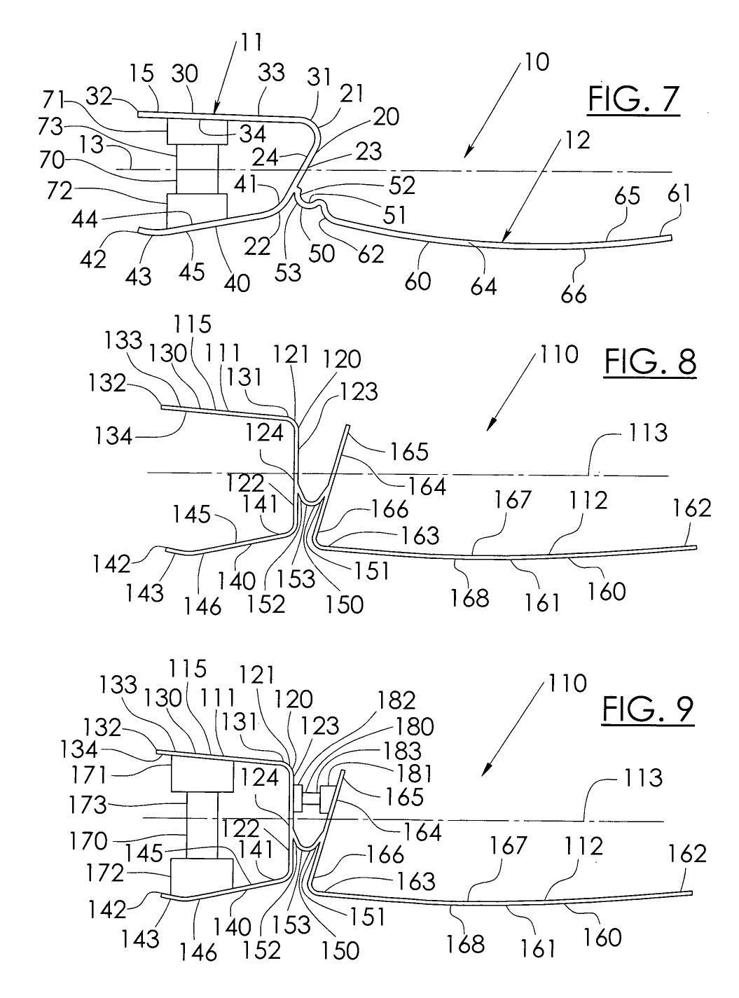

Turning now to FIG. 7, a stiffener 70 can optionally be provided. The stiffener 70 can have a first end 71 and a second end 72, and can have a compressible component 73 between the ends. The first end 71 can be attached to the bottom surface 34 of the attachment platform 30, and the second end 72 can be attached to the top surface 44 of the heel strike 44. The stiffener 70 can be used to reduce the amount of deflection by the attachment platform 30 and the heel strike 40. The compressible component 73 can be a mechanically adjustable compressible component. An example of a potential compressible component comprises a linear spring component wherein the spring can be manually pre-compressed a selected distance to provide a selected amount of resistance to the flexing of the attachment platform 20 and heel strike 40. Also, a compressible fluid could be contained within the compressible component 73, such that the compressibility of the component could be related to the amount of fluid within the component. The compressible component could alternatively be non-adjustable, and instead be interchangeable wherein a component with selected characteristics is inserted between the ends 71 and 72.

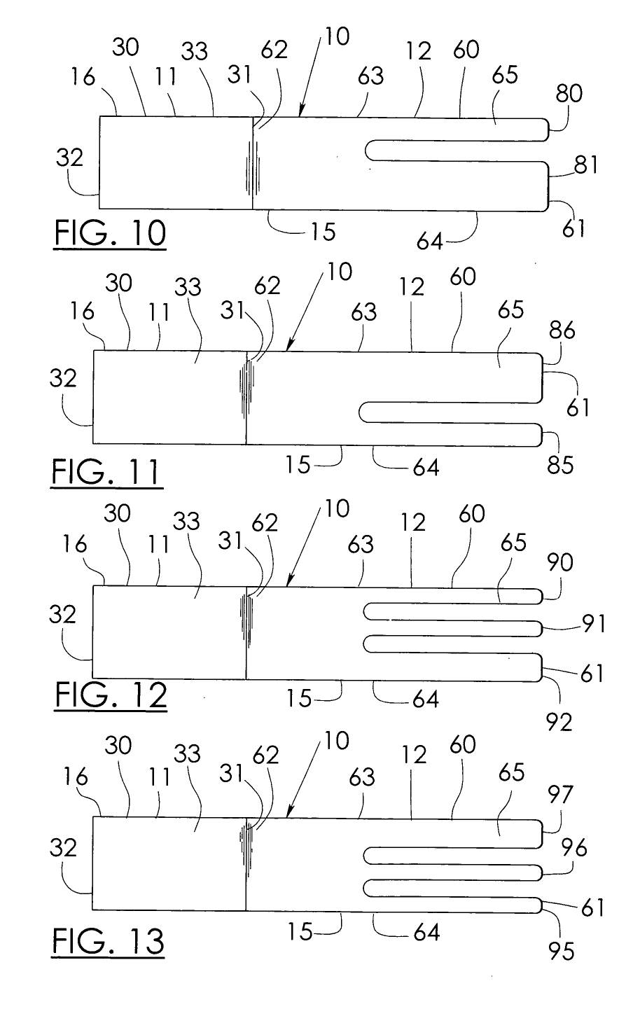

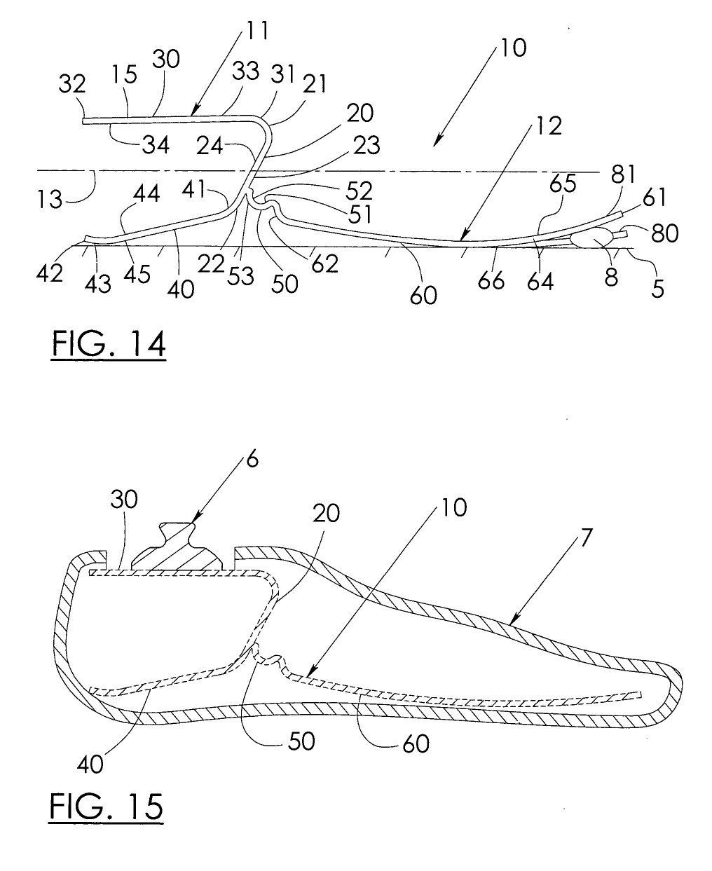

Looking now to FIGS. 10-14, several alternative embodiments of the present invention are shown. In FIG. 10, two toes 80 and 81 are formed into the toe plate 60. Toe 80 is on the left side 16 of the foot 10, and toe 81 is on the rights side 15 of the foot. Toe 81 is about two times as wide as toe 80 in this embodiment. The toes 80 and 81 can flexibly deflect different amounts depending on what terrain the person encounters. This is illustrated in FIG. 14, wherein an object 8 is located under toe 81, and toe 80 rests on the ground 5. This advantageously adds flexibility to the foot 10, and makes the person have greater stability on the terrain. Yet, both toes 80 and 81 will deflect the same amount on flat surfaces.

The foot shown in FIG. 11 is the opposite of the foot shown in FIG. 10. The foot in FIG. 11 has toes 85 and 86. Toe 85 is located on the left side 16 of the foot 10, and toe 86 is on the right side 15 of the foot. Toe 86 is approximately twice as wide as toe 85. The feet illustrated in FIGS. 10 and 11 could be left and right prosthetic feet, respectively.

FIG. 12 illustrates a further embodiment of the present invention. In this embodiment, the foot has toes 90, 91 and 92. Toe 90 is located on the left side 16 of the foot 10, toe 92 is located on the right side 15 of the foot, and toe 91 is located between toes 90 and 92. Toes 90 and 91 are generally about the same size, and toe 92 is generally wider than toes 90 and 91.

The foot shown in FIG. 13 is the opposite of the foot shown in FIG. 12. The foot 10 shown in this figure has toes 95, 96 and 97. Toe 97 is located on the left side 16 of the foot 10, toe 95 is located on the right side 15 of the foot, and toe 96 is located between toes 95 and 97. Toes 95 and 96 are generally about the same size, and toe 97 is generally wider than toes 95 and 96. The feet illustrated in FIGS. 12 and 13 could be left and right prosthetic feet, respectively.

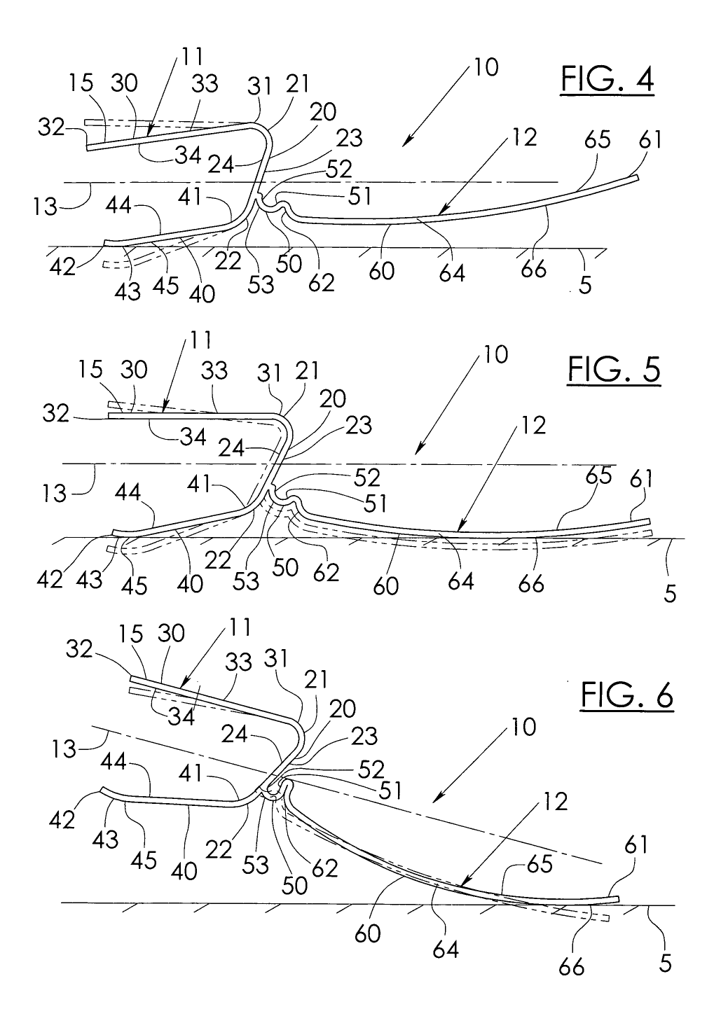

Operation of the present invention is illustrated in FIGS. 4-6. FIG. 4 is illustrative of load conditions at heel-strike. At heel-strike, the heel strike 40 makes initial contact with the ground 5, and the front foot portion 12 is off the ground completely. The foot rear portion 11 compresses, as the attachment platform 30 flexibly deflects downward and the heel strike 40 flexibly deflects upward. The deflection of these two components is severe, especially when the person is heavy or when the person runs or jumps, or otherwise has a lot of momentum that needs to be absorbed by the foot 10. Energy is stored in the deflected attachment platform 30 and heel strike 40. The toe plate 60 and momentum interrupter 50 do not absorb or store any energy at heel-strike.

As the person moves toward mid-stance, the heel strike 40 releases some of its stored energy to assist in propelling the foot 10 to the mid-stance position. Also, the attachment platform 30 releases some of its stored energy to assist in raising the person upwards to normal height.

An operational view at mid-stance is provided in FIG. 5. At mid-stance, the heel strike 40 and the toe plate 60 both contact the ground. The attachment platform 30, the heel strike 40, and the toe plate 60 are all moderately deflected. Further, the momentum interrupter 50 is moderately compressed. If the person chooses to rock back onto the heel strike 40, the momentum interrupter 50 decompresses, or expands, and the toe plate deflexes to assist the person in rocking backwards. Conversely, if the person chooses to move towards toe-off, the heel strike 40 releases energy to assist the person towards toe-off.

It is noteworthy, that if the person happens to land in a flat foot orientation, the attachment platform 30, the heel strike 40 and the toe plate 60 may all deflect severely, and the momentum interrupter 50 may compress severely, while absorbing the shock, and then release some energy to return the foot to mid-stance equilibrium.

FIG. 6 shows an operational view of the present invention at toe-off, further illustrating the forefoot momentum interruption of the present invention. At toe-off, the front end 61 of the toe plate 60 is the only component that is contacting the ground 5. The toe plate 60 is severely deflected and the momentum interrupter 50 is severely compressed. The entire rear foot portion 11 is slightly moved forward with respect to the toe plate 60 at toe-off, due to the compression of the momentum interrupter 50. Further, the attachment platform 30 is moderately deflected upward. The heel strike 40 is not deflected during toe-off.

At toe-off, the toe plate 60 releases energy that pushes the foot in a direction generally perpendicular to the bottom surface 66 of the toe plate 60. The momentum interrupter 50 decompresses, or expands, to release energy that assists in pushing the rear foot portion 11 away from the toe plate 60. This motion is offset from the motion caused by the toe plate 60.

The compression of the momentum interrupter 50 disrupts the flow of energy, and the velocity of the foot during toe-off. These disruptions occur because the compression occurs along the compression axis which is out of sync with the remainder of the energy flow and motion of the foot. In one embodiment, the compression axis is nearly parallel with the longitudinal axis 13 of the foot 10. The shortening of the momentum interrupter 50 during compression temporarily shortens the overall length of the front foot portion 12 at toe-off. The momentum interrupter then expands during the release of the energy, and the overall length of the foot returns to its original length.

Looking now at FIG. 7, an alternative preferred embodiment of the present invention is shown. Accordingly, a foot I 10 is provided that is generally comprised of a rear foot portion 111 and a front foot portion 12. The rear foot portion 111, or rear portion, is generally comprised of a riser panel 120, an attachment platform 130 and a heel strike 140. The front foot portion 112 is generally comprised of a momentum interrupter 150 and a toe plate 160. The prosthetic foot has a longitudinal axis 113 spanning the length of the foot 110.

The riser panel 120 has a top end 121 and a bottom end 122. The riser panel 120 also has a front surface 123 and a rear surface 124. In the preferred embodiment, the riser panel 121 is generally vertically oriented with the top end 121 being located over the bottom end 122. However, other orientations of the riser panel 120 could be used without departing from the broad aspects of the present invention. The riser panel 120 is flexible along its length.

The attachment platform 130 has a front end 131 and a rear end 132. The attachment platform 130 also has a top surface 133 and a bottom surface 134. The front end 131 of the attachment platform 130 is preferably integrally connected to the top end 121 of the riser panel 120, such that the rear end 132 is distal from the riser panel 120. This connection is preferably a rounded connection wherein sharp edges are eliminated. The attachment platform 130 flexibly depends rearward from the riser panel 120. The attachment platform 130 is preferably angled slightly upward so that the rear end 132 is slightly higher that the front end 131. During a downward load condition, the attachment platform 130 flexibly deflects downward in response to an applied force. In this regard, the rear end 132 deflects down in relation to the front end 131. Conversely, during an upward load condition, the attachment platform 130 flexibly deflects upward in response to the applied force. In both situations, the attachment platform 130 stores and then releases the stored energy when the load condition is ended.

The heel strike 140 has a front end 141 and a rear end 142. A lip 143 can be at the rear end 142 of the heel strike 140. The heel strike 140 further has a top surface 144 and a bottom surface 145. The heel strike 140 is preferably integrally connected to the bottom end 122 of the riser panel 120, such that the rear end 142 is distal from the riser panel 120. This connection is preferably a rounded connection wherein sharp edges are eliminated. The heel strike 140 flexibly depends rearward from the riser panel 120. The heel strike 140 preferably depends downward from the riser panel 120, such that the heel strike rear end 142 is lower than the heel strike front end 141. The lip 143 can be angled with respect to the remainder of the heel strike 140 such that it lies in a plane that is close to parallel to the longitudinal axis 113 in a no load condition. During a load condition, the heel strike 140 deflects upward in response to an applied force. In this regard, the rear end 142 deflects upward in relation to the front end 141. The heel strike 140 stores and then releases the stored energy when the load condition is ended.

A momentum interrupter 150 is advantageously provided according to the present invention. A preferred alternative momentum interrupter 150 has a front 151, a rear 152 and a bottom 153. In this regard, the momentum interrupter 150 generally has a U-shape. However, other shapes can be provided without departing from the broad aspects of the present invention. The momentum interrupter 150 is preferably integrally connected to the riser panel 120, wherein the momentum interrupter rear 152 is integral with the riser panel front surface 123. In the illustrated embodiment, the momentum interrupter 150 is shown connected near the bottom end 122 of the riser panel 120. However, other orientations can exist without departing from the broad aspects of the present invention. The momentum interrupter has a length between the front 151 and rear 152. During a load condition, the momentum interrupter front 151 compresses towards the momentum interrupter rear 152, and the momentum interrupter length is decreased. This compression preferably occurs along a compression axis. The momentum interrupter 150 stores and then releases the stored energy when the load condition is ended.

The toe plate 160 has a forward section 161 and a rear wall 164. The forward section has a front end 162 and a rear end 163. The rear wall 164 has a top 165 and a bottom 166. The toe plate 160 has a top surface 167 and a bottom surface 168. The toe plate 160 is preferably integrally connected to the momentum interrupter 150. In this regard, the front 151 of the momentum interrupter is connected to the rear wall 164 between the top 165 and the bottom 166. The toe plate forward section 161 preferably is slightly upwardly curved along its length. The toe plate 160 flexibly depends forwardly and preferably slightly downwardly from the momentum interrupter 150. During a load condition, the front end 162 of the toe plate forward section 161 flexes upward in relation to the rear end 163. The toe plate 160 stores and then releases the stored energy when the load condition is ended.

Turning now to FIG. 9, a stiffener 170 can optionally be provided. The stiffener 170 can have a first end 171 and a second end 172, and can have a compressible component 173 between the ends. The first end 171 can be attached to the bottom surface 134 of the attachment platform 130, and the second end 172 can be attached to the top surface 144 of the heel strike 140. The stiffener 170 can be used to reduce the amount of deflection by the attachment platform 130 and the heel strike 140. The compressible component 173 can be a mechanically adjustable compressible component. The compressible component could alternatively be non-adjustable, and instead be interchangeable wherein a component with selected characteristics is inserted between the ends 171 and 172.

A second stiffener 180 can also optionally be provided. The stiffener 180 can have a first end 181 and a second end 182, and can have a compressible component 183 between the ends. The first end 181 can be attached to the front surface 123 of the riser platform 120, and the second end 182 can be attached to the rear surface of the rear wall 164. The stiffener 180 can be used to reduce the amount of compression in the momentum interrupter 150. The compression element 183 can be of the same type as compression element 173.

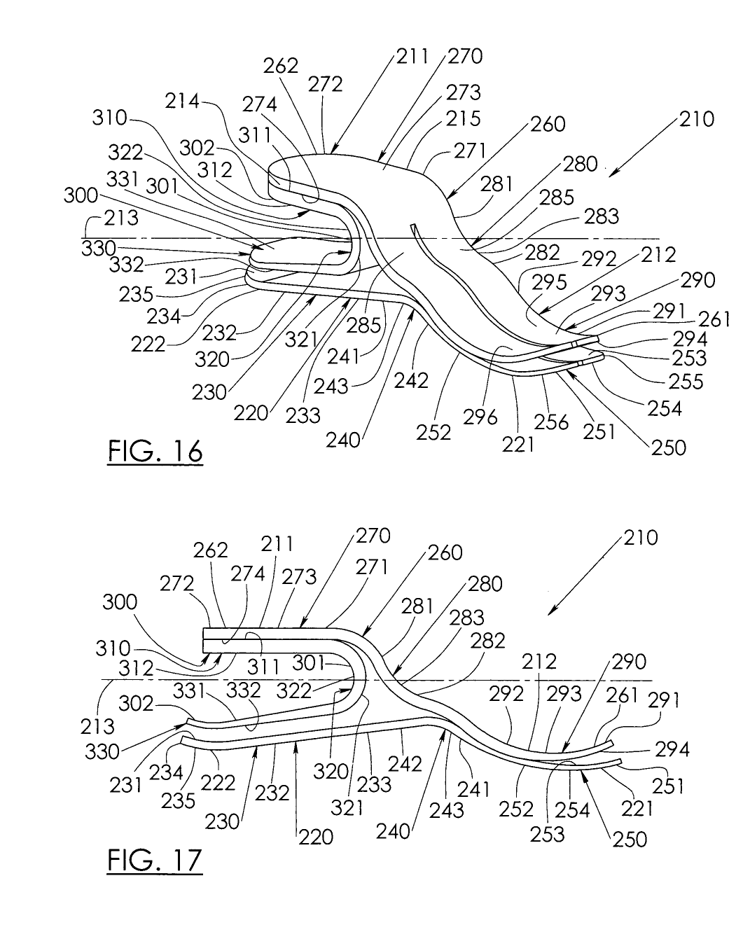

Looking now to FIGS. 16 and 17, a further alternative embodiment of the present invention is shown. Accordingly, a prosthetic foot 210 is provided that is generally comprised of a rear foot portion 211 and a front foot portion 212. The prosthetic foot 210 has a longitudinal axis 213 spanning the length of the foot 210. The foot 210 has a right side 214 and a left side 215.

The prosthetic foot 210 is comprised of a base plate 220 having a base plate front 221 located in the foot front portion 212, and a base plate rear 222 located in the rear portion 211 of the prosthetic foot 210. The prosthetic foot 210 is further comprised of a momentum interrupting component 260 having a momentum interrupting component front 261 located in the foot front portion 212, and a momentum interrupting component rear 262 located in the rear portion 211 of the prosthetic foot 210.

Looking more closely at the base plate 220, it is shown that the base plate is preferably comprised of a heel strike 230, a toe plate 250, and an arch 240 located between the heel strike and toe plate.

The heel strike 230 has a top surface 231 and a bottom surface 232. The heel strike 230 further has a front end 233 and a rear end 234. A lip 235 can be at the rear end 234 of the heel strike 230. The lip 235 can be angled with respect to the remainder of the heel strike 230 such that it lies in a plane that is close to parallel to the longitudinal axis 213 in a no load condition. During a load condition, the heel strike 230 flexibly deflects upward in response to an applied force. In this regard, the rear end 234 deflects upward in relation to the front end 233. The heel strike 230 stores and then releases the stored energy when the load condition is ended.

The arch 240 has a front 241 and a rear 242. The arch 240 further also has a peak 243 between the front 241 and the rear 242. The peak 243 of the arch 240 is preferably located above the front 241 and the rear 242. During a loading condition, the arch 240 can expand, such that the arch front 241 can be spread from the arch rear 242. The rear 242 is preferably integrally connected to the front 233 of the heel strike 230. In a further alternative embodiment of the present invention, the arch 240 is split into two components.

The toe plate 250 has a front 251 and a rear 252. The toe plate 250 has a top surface 253 and a bottom surface 254. The toe plate 250 is preferably integrally connected to the arch 240. In this regard, the front 241 of the arch 240 is connected to the rear 252 of the toe plate 250. The toe plate preferably has a slightly upwardly curved profile along its length. During a load condition, the front end 251 of the toe plate 250 flexibly deflects upward in relation to the rear end 252. The toe plate 250 stores and then releases the stored energy when the load condition is ended. As a further alternative, the toe plate 250 can be split into two toes 255 and 256, respectively. The two toes 255 and 256 are shown having similar widths. Yet, it is understood that the toes 255 and 256 could have different respective widths without departing from the broad aspects of the present invention.

Looking more closely at the momentum interrupting component 260 now, it is shown that the momentum interrupting component is preferably comprised of an attachment platform 270, a momentum interrupter 280, and a toe reinforcement component 290.

The attachment platform 270 has a front end 271 and a rear end 272. The attachment platform 270 also has a top surface 273 and a bottom surface 274. During a downward load condition, the attachment platform 270 flexibly deflects downward in response to an applied force. In this regard, the rear end 272 deflects down in relation to the front end 271. Conversely, during an upward load condition, the attachment platform 270 flexibly deflects upward in response to the applied force. In both situations, the attachment platform 270 stores and then releases the stored energy when the load condition is ended.

The momentum interrupter 280 is advantageously provided according to the present invention. A further preferred alternative momentum interrupter 280 has a rear 281, a front 282 and a bottom 283, or base. In this regard, the momentum interrupter 280 generally has a U-shape. However, other shapes can be provided without departing from the broad aspects of the present invention. The momentum interrupter 280 is preferably integrally connected to attachment platform 270, wherein the momentum interrupter rear 281 is integral with the attachment platform front 271. The momentum interrupter has a length between the front 282 and rear 281. During a load condition, the momentum interrupter front 282 compresses toward the momentum interrupter rear 281, and the momentum interrupter length is decreased. This compression preferably occurs along a compression axis. The momentum interrupter 280 stores and then releases the stored energy when the load condition is ended.

The toe reinforcement component 290 has a front 291 and a rear 292. The toe reinforcement member further has a top surface 293 and a bottom surface 294. The toe reinforcement component 290 is preferably integrally connected to the momentum interrupter 280, such that the rear 292 of the toe reinforcement component 290 is connected to the front 282 of the momentum interrupter. As a further alternative, the toe reinforcement component 290 can be split into two components 295 and 296, or members, respectively. The two components 295 and 296 are shown having similar widths. Yet, it is understood that the components 295 and 296 could have different respective widths without departing from the broad aspects of the present invention.

It is understood that the momentum interrupting component 260 is generally located above the base plate 220. In this regard, the attachment platform 270 is generally located a predetermined distance above the heel strike 230. The rear 292 of the toe reinforcement member 290 is preferably connected to the rear 252 of the toe plate 250. The front 291 of the toe reinforcement member 290 is preferably located a predetermined distance above the front 251 of the toe plate 250.

A vertical shock absorbing component 300 can optionally be provided for use in connection with the prosthetic foot 210. The vertical shock absorbing component has a front 301 and a rear 302. A top piece 310 having a top surface 311 and a bottom surface 312 is provided. A front piece 320 having a front surface 321 and a rear surface 322 is also provided. A bottom piece 320 having a top surface 331 and a bottom surface 332 is further provided. The top piece 310, front piece 320 and bottom piece 330 are preferably integrally connected. The rear 302 of the vertical shock absorbing component 300 is preferably open. The vertical shock absorbing component 300 is preferably located in the rear foot portion 211 of the prosthetic foot 210. The top surface 311 of the top piece 310 is preferably connected to the bottom surface 274 of the attachment platform. The bottom surface 332 of the bottom piece 330 is preferably spaced a predetermined distance above the top surface 231 of the heel strike 230.

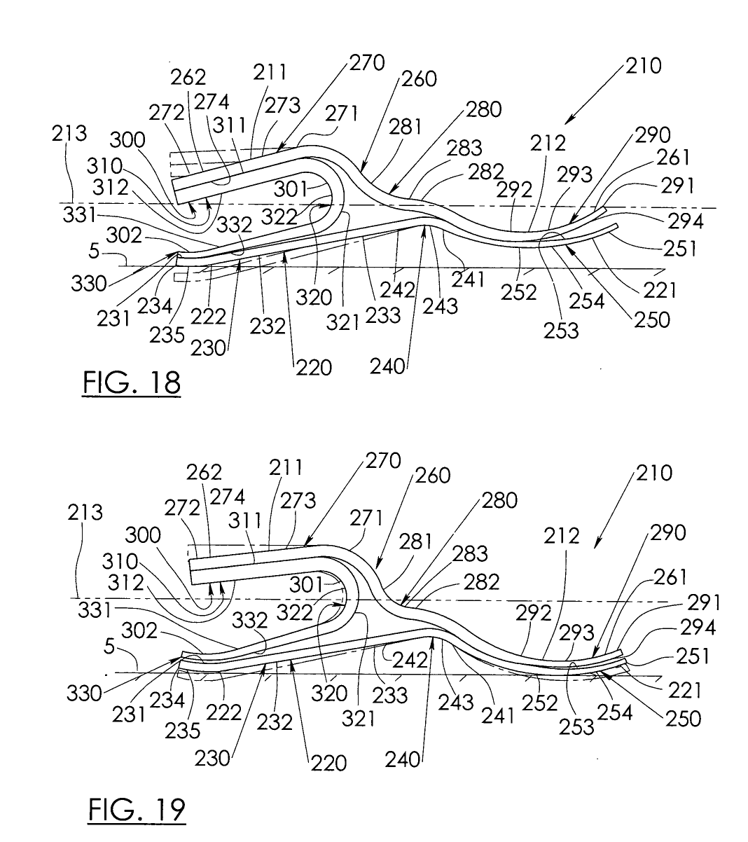

The operation of this alternative embodiment is illustrated in FIGS. 18-20. FIG. 18 is illustrative of load conditions at heel-strike. At heel-strike, the heel strike 230 makes initial contact with the ground 5, and the front foot portion 212 is off the ground completely. The foot rear portion 211 compresses, as the attachment platform 270 flexibly deflects downward and the heel strike 230 flexibly deflects upward. The deflection of these two components is severe, especially when the person is heavy or when the person runs or jumps, or otherwise has a lot of momentum that needs to be absorbed by the foot 210. Energy is stored in the deflected attachment platform 270 and heel strike 230. The vertical shock absorbing component 300 can absorb energy in increased load conditions. In this regard, the top piece 310 can flexibly deflect downward with the attachment platform 270. Further, once the heel strike 230 flexibly deflects a predetermined amount, the top surface 231 contacts the bottom surface 332 of the bottom piece 330 of the vertical shock absorbing component 300. The bottom piece 330 then flexibly deflects as the heel strike 230 further flexibly deflects. It can be appreciated that the thickness of the components, and the spacing between the heel strike 230 and vertical shock absorbing component 300 can be adjusted to suit a set of parameters without departing from the broad aspects of the present invention.

As the person moves toward mid-stance, the heel strike 230 releases some of its stored energy to assist in propelling the foot 210 to the mid-stance position. Also, the attachment platform 270 releases some of its stored energy to assist in raising the person upwards to normal height.

An operational view at mid-stance is provided in FIG. 19. At mid-stance, the heel strike 230 and the toe plate 250 both contact the ground. The attachment platform 270, the heel strike 230, and the toe plate 250 are all moderately deflected. Further, the momentum interrupter 280 is moderately compressed. If the person chooses to rock back onto the heel strike 230, the momentum interrupter 280 decompresses, or expands, and the toe plate 250 deflexes to assist the person in rocking backwards. Conversely, if the person chooses to move towards toe-off, the heel strike 230 releases energy to assist the person towards toe-off.

It is noteworthy, that if the person happens to land in a flat foot orientation, the attachment platform 270, the heel strike 230 and the toe plate 250 may all deflect severely, and the momentum interrupter 280 may compress severely, while absorbing the shock, and then release some energy to return the foot to mid-stance equilibrium.

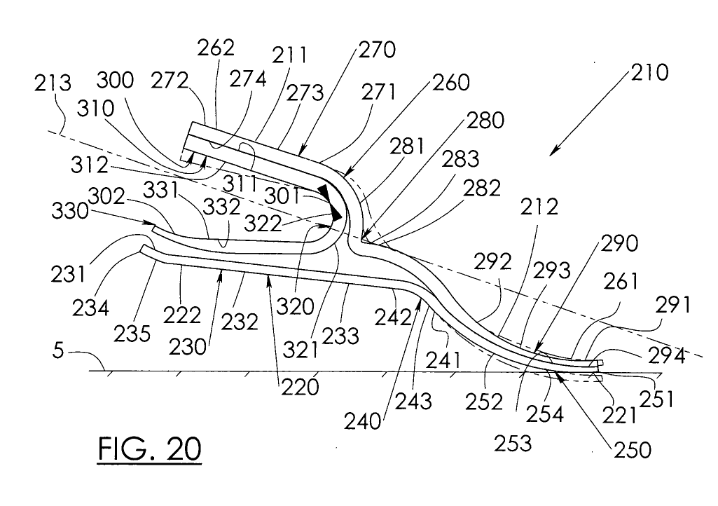

FIG. 20 shows an operational view of the present invention at toe-off, further illustrating the forefoot momentum interruption of the present invention. At toe-off, the front end 251 of the toe plate 250 is the only component that is contacting the ground 5. The toe plate 250 is severely deflected and the momentum interrupter 280 is severely compressed. The rear portion 262 of the momentum interrupting component 260 is moved forward with respect to front portion 261 at toe-off, due to the compression of the momentum interrupter 280. It is shown that the top surface 253 of the toe plate 250 contacts the bottom surface 294 of the toe reinforcement component 290 after the toe plate 290 flexibly deflects upward a predetermined amount. After the toe plate 290 flexibly deflects the predetermined amount, the toe reinforcement member 290 will flexibly deflect upward along with the toe plate 250. Further, the attachment platform 270 is moderately deflected upward during toe-off. The heel strike 230 is not deflected during toe-off.

At toe-off, the toe plate 250 releases energy that pushes the foot in a direction generally perpendicular to the bottom surface 254 of the toe plate 250. The momentum interrupter 280 decompresses, or expands, to release energy that assists in pushing the rear foot portion 211 away from the toe plate 250. This motion is offset from the motion caused by the toe plate 250.

The compression of the momentum interrupter 280 disrupts the flow of energy, and the velocity of the foot during toe-off. These disruptions occur because the compression occurs along the compression axis which is out of sync with the remainder of the energy flow and motion of the foot. The shortening of the momentum interrupter 280 during compression temporarily shortens the overall length of the momentum interrupting component 260 at toe-off. The momentum interrupter then expands during the release of the energy, and the overall length of the momentum interrupting component 260 returns to its original length.

Thus it is apparent that there has been provided, in accordance with the invention, a prosthetic foot that fully satisfies the objects, aims and advantages as set forth above. While the invention has been described in conjunction with specific embodiments thereof, it is evident that many alternatives, modifications, and variations will be apparent to those skilled in the art in light of the foregoing description. Accordingly, it is intended to embrace all such alternatives, modifications, and variations as fall within the spirit and broad scope of the appended claims.

I claim:

1. A prosthetic foot comprising:
a rear foot portion deflectably contactable against a surface and connectable to a prosthetic component; and
a front foot portion connected to said rear foot portion, said front foot portion comprising:
a momentum interrupter that is compressible; and
a toe plate that is flexible,
wherein during toe-off conditions, said momentum interrupter compresses and said toe plate flexes.

2. The prosthetic foot of claim 1 wherein said rear foot portion comprises:
a riser panel;
an attachment platform depending rearward from said riser panel;
a heel strike depending rearward from the riser panel, said heel strike being located below said attachment platform.

3. The prosthetic foot of claim 2 further comprising a stiffener, said stiffener being located between said attachment platform and said heel strike.

4. The prosthetic foot of claim 3 wherein said stiffener is selectably adjustable.

5. The prosthetic foot of claim 2 wherein said riser panel has a riser panel top and a riser panel bottom, wherein said riser panel top is located forward of said riser panel bottom.

6. The prosthetic foot of claim 1 wherein said momentum interrupter comprises a momentum interrupter back and a momentum interrupter front, and during toe-off conditions, said momentum interrupter front compresses towards said momentum interrupter back.

7. The prosthetic foot of claim 1 wherein said prosthetic foot is made from carbon epoxy composite material.

8. The prosthetic foot of claim 1 wherein said toe plate comprises a plurality of toes.

9. The prosthetic foot of claim 1 wherein:
said rear foot portion comprises:
a heel strike for deflectably contacting against a surface;
an attachment platform for being connectable to a prosthetic component;
said toe plate and said heel strike being integral parts of a base plate; and
said attachment platform and said momentum interrupter being integral parts of a momentum interrupting component that is located above said base plate.

10. The prosthetic foot of claim 9 wherein said momentum interrupting component further comprises a toe reinforcement component located above said toe plate.

11. The prosthetic foot of claim 10 wherein:
said toe plate comprises a plurality of toes; and
said toe reinforcement component comprises a plurality of toe reinforcement members located above said plurality of toes.

12. The prosthetic foot of claim 9 wherein said rear foot portion further comprises a vertical shock absorption component attached to said attachment platform and spaced a predetermined distance from said heel strike.

13. A prosthetic foot comprising:
a riser panel;
an attachment platform depending rearward from said riser panel;
a heel strike depending rearward from the riser panel, said heel strike being located below said attachment platform;
a momentum interrupter having a momentum interrupter front and a momentum interrupter back, said momentum interrupter back being connected to said riser panel; and
a toe plate being connected to said momentum interrupter and extending forward there from.

14. The prosthetic foot of claim 13 wherein:
said riser panel has a riser panel top and a riser panel bottom;
said attachment platform depends rearward from said riser panel top; and
said heel strike depends rearward from said riser panel bottom.

15. The prosthetic foot of claim 14 wherein said riser panel is a forward leaning riser panel, such that said riser panel top is located forward of said riser panel bottom.

16. The prosthetic foot of claim 13 wherein during toe-off conditions, said momentum interrupter front compresses towards said momentum interrupter back, and said toe plate flexes.

17. The prosthetic foot of claim 13 wherein:
said attachment platform is integrally connected to said riser panel;
said heel strike is integrally connected to said riser panel;
said momentum interrupter is integrally connected to said riser panel; and
said toe plate is integrally connected to said momentum interrupter.

18. The prosthetic foot of claim 17 wherein said prosthetic foot is made from carbon epoxy composite material.

19. The prosthetic foot of claim 13 wherein said toe plate comprises a plurality of toes.

20. The prosthetic foot of claim 13 further comprising a stiffener, said stiffener being located between said attachment platform and said heel strike.

21. The prosthetic foot of claim 20 wherein said stiffener is selectably adjustable.

22. A prosthetic foot comprising:
a rear foot portion having:
a riser panel;
an attachment platform located rearward of said riser panel;
a heel strike located below said attachment platform; and
a front foot portion connected to said rear foot portion, wherein said front foot portion has a length with a first part and a second part, and is compressible at said first part of said length and is flexible at said second part of said length.

23. The prosthetic foot of claim 22 wherein:
said riser panel has a riser panel top and a riser panel bottom;
said attachment platform depends rearward from said riser panel top; and
said heel strike depends rearward from said riser panel bottom.

24. The prosthetic foot of claim 22 wherein said front foot portion comprises is an integrally formed front foot portion, and:
a momentum interrupter is located in said first part of said length, said momentum interrupter having a momentum interrupter front and a momentum interrupter rear; and
a foot plate located in said second part of said length,
wherein during toe-off conditions, said momentum interrupter front compresses towards said momentum interrupter back, and said toe plate flexes.

25. The prosthetic foot of claim 24 wherein said prosthetic foot is made from carbon epoxy composites.

26. The prosthetic foot of claim 24 wherein said toe plate comprises a plurality of toes.

27. The prosthetic foot of claim 22 further comprising a stiffener, said stiffener being located between said attachment platform and said heel strike.

28. The prosthetic foot of claim 27 wherein said stiffener is adjustable.

29. The prosthetic foot of claim 22 wherein said riser panel is a forward leaning riser panel.

30. The prosthetic foot of claim 22 wherein said momentum interrupter is integrally connected to said riser panel.

31. The prosthetic foot of claim 22 further comprising:
a momentum interrupting component with a momentum interrupting component front and a momentum interrupting component rear, wherein said attachment platform is located in said momentum interrupting component rear, said momentum interrupting component further comprising a momentum interrupter located at said first part of said front foot portion; and
a base plate comprising a base plate rear and a base plate front, wherein said heel strike is located at said base plate rear, said base plate further comprising a toe plate at said second end of said front foot portion.

32. The prosthetic foot of claim 31 further comprising a vertical shock absorbing component, wherein said riser panel is a front component of said vertical shock absorbing component, said vertical shock absorbing component further comprising a top connected to said attachment platform and a bottom spaced a predetermined distance above said heel strike.

33. The prosthetic foot of claim 31 wherein said momentum interrupting component further comprises a toe reinforcement component above said toe plate.

34. The prosthetic foot of claim 31 wherein said base plate further comprises an arch between said heel strike and said toe plate.

35. A prosthetic foot comprising:
a base plate comprising:
a heel strike; and
a toe plate forward of said heel strike; and
a momentum interrupting component comprising:
an attachment platform located over said heel strike; and
a momentum interrupter forward of said attachment platform and rearward of said toe plate.

36. The prosthetic foot of claim 35 wherein said base plate further comprises an arch between said heel strike and said toe plate.

37. The prosthetic foot of claim 35 wherein said momentum interrupting component further comprises a toe reinforcement component above said toe plate.

38. The prosthetic foot of claim 37 wherein said toe plate comprises a plurality of toes and said toe reinforcement component comprises a plurality of toe reinforcement components.

39. The prosthetic foot of claim 35 further comprising a vertical shock absorbing component connected to said attachment platform and being a predetermined distance above said heel strike.
